SEQUENCE LISTING (1 sequence)

SEQ ID NO: 1 - protein, 5 aa, Artificial Sequence
  TMTP1
NVVRQ

(19) United States
(12) Patent Application Publication
(10) Pub. No.: US 2020/0206358 A1
(43) Pub. Date: Jul. 2, 2020

(54) LONG-CIRCULATING THERANOSTIC AGENTS FOR DIAGNOSING AND IMAGING METASTATIC TUMORS

(71) Applicant: The Board of Trustees of the Leland Stanford Junior University, Stanford, CA (US)

(72) Inventors: Yesen Li, Palo Alto, CA (US); Zhen Cheng, Palo Alto, CA (US)

(21) Appl. No.: 16/719,615

(22) Filed: Dec. 18, 2019

Related U.S. Application Data
(60) Provisional application No. 62/891,144, filed on Aug. 23, 2019.

(30) Foreign Application Priority Data
Dec. 18, 2018 (CN) 201811549217.2

(51) Int. Cl. A61K 47/68; A61K 47/64; A61K 49/00; A61K 49/08; A61P 35/04; A61K 51/04

(52) U.S. Cl. CPC A61K 47/6803; A61K 47/6455; A61K 49/0002; A61K 45/06; A61P 35/04; A61K 51/0482; A61K 49/085

(57) ABSTRACT

Theranostic agents useful for imaging and treating metastatic cancer are provided. The theranostic agents comprise a TMTP1 peptide conjugated to an albumin binding moiety to prolong circulation lifetime and a chelating agent to allow complexation of a diagnostic metal ion with the theranostic agent. The theranostic agent can be conjugated, for example, to a positron-emitting metal radionuclide suitable for PET imaging such as ⁶⁴Cu, ⁶⁸Ga, ⁴⁴Sc, ⁸⁶Y, ⁸⁹Zr, or ⁸²Rb; or a gamma-emitting metal radionuclide suitable for single photon emission computed tomography (SPECT) imaging such as ⁶⁷Ga, ^{99m}Tc, ¹¹¹In, or ¹⁷⁷Lu. Alternatively, theranostic agents can be conjugated with a paramagnetic metal ion suitable for use in magnetic resonance imaging (MRI) such as manganese (e.g., Mn²⁺), iron (e.g., Fe³⁺, Fe²⁺) or gadolinium (e.g., Gd³⁺). Such theranostic agents show selective uptake by metastatic cancer cells and are useful for PET, SPECT, or MRI imaging of metastatic cells in vitro and in vivo.

### BACKGROUND

Approximately 90% of cancer-related deaths are a result of cancers reaching the metastatic stage, where cells from the primary tumor begin to migrate through the vasculature and form new tumors throughout the body. Once cancers reach this stage, the available treatment options are scarce and largely ineffective, and as a result the median survival time for patients is on the order of 1-2 years. The earlier carcinoma metastasis is diagnosed, the better chance a patient has of surviving. Accurate detection, diagnosis, and staging may lead to more appropriate treatments and improved clinical outcomes. Thus, there is a dire need for improved diagnostic methods and therapies that specifically target the mechanisms of metastasis to reduce mortality caused by cancer.

Theranostics is a field of medicine that combines specific targeted therapy based on specific targeted diagnostic tests. The theranostics paradigm can use nanoscience to unite diagnostic and therapeutic applications to form a single agent, allowing for diagnosis, drug delivery and treatment response monitoring. The development of molecular diagnostic tests and targeted therapeutics in an interdependent and collaborative manner, focusing on individualized treatment by targeting therapy to an individual's specific disease subtype and genetic profile, enables optimization of drug efficacy and safety, assisting in streamlining the drug development process. This information allows decisions to be made on timing, quantity, type of drugs, and choice of treatment procedure, as well as helping to evaluate a patient's response to treatment.

Molecular imaging with positron emission tomography (PET) using tumor-seeking radiolabeled-molecules has gained wide acceptance in oncology, allowing earlier diagnosis and better clinical management of cancer patients (Gambhir S. S. (2002) Nat. Rev. Cancer 2: 683-693; Weissleder R. (2002) Nat. Rev. Cancer 2: 11-18; Fletcher et al., (2008) J. Nucl. Med. 49: 480-508). A variety of molecules, including glucose analogues, monoclonal antibodies (mAbs), antibody fragments, and peptides, can be used as tumor-seeking molecules with different levels of tumor accessibility and specificity. Earlier detection of metastatic cancer is crucial and may significantly improve outcome. Therefore, there is a continuing need to develop new molecular imaging techniques to improve the sensitivity and specificity of cancer detection.

### SUMMARY

Theranostic agents useful for targeted imaging and treatment of metastatic cancer are provided. The theranostic agents comprise a TMTP1 peptide conjugated to an albumin binding moiety and a chelating agent. The TMTP1 peptide confers selectivity for metastatic cancerous cells. The presence of a chelating agent in the theranostic agent allows complexation with a diagnostic metal ion. For example, the theranostic agent can be conjugated to a positron-emitting metal radionuclide suitable for PET imaging such as ⁶⁴Cu, ⁶⁸Ga, ⁴⁴Sc, ⁸⁶Y, or ⁸²Rb; or a gamma-emitting metal radionuclide suitable for single photon emission computed tomography (SPECT) imaging such as ⁶⁷Ga, ^{99m}Tc, ¹¹¹In, or ¹⁷⁷Lu. Alternatively, theranostic agents can be conjugated with a paramagnetic metal ion suitable for use in magnetic resonance imaging (MRI) such as manganese (e.g., Mn²⁺), iron (e.g., Fe³⁺, Fe²⁺) or gadolinium (e.g., Gd³⁺). The theranostic agent may also be labeled with a positron-emitting radiohalogen such as ¹²⁴I or ¹⁸F suitable for PET imaging or a gamma-emitting radiohalogen such as ¹³¹I, ¹²⁵I, or ¹²³I suitable for SPECT imaging. In addition, the theranostic agent can be conjugated with radionuclides suitable for radiotherapy such as ¹⁷⁷Lu, ⁹⁰Y, ²¹³Bi, ²¹²Pb, ²¹¹At, ²²⁵Ac, ¹⁶⁶Ho, ⁸⁹Sr, ¹⁵³Sm, ²²³Ra, ²²⁶Ra, ¹³⁷Cs, ¹⁹⁸Au, ¹⁸²Ta, ¹⁹²Ir, ¹²⁵I, or ¹³¹I. Such theranostic agents show selective binding to metastatic cancer cells and accumulation in metastatic tumors, and are useful for PET, SPECT, or MRI imaging and radionuclide treatment of metastatic cells in vitro and in vivo.

In certain embodiments, the albumin binding moiety is an Evans blue dye (tetrasodium (6E,6′E)-6,6-[(3,3′-dimethylbiphenyl-4,4′-diyl)di(1E) hydrazin-2-yl-1-ylidene]bis(4-amino-5-oxo-5,6-dihydronaphthalene-1,3-disulfonate)), or an Evans blue derivative such as a truncated Evans blue (tEB) dye, wherein a 1-amino-naphthol-2,4-disulfonic acid moiety of Evans blue is replaced with the chelating agent to allow metal complexation to the theranostic agent.

In certain embodiments, the chelating agent is selected from the group consisting of 1,4,7,10-tetraazacyclododecane-1,4,7,10-tetraacetic acid (DOTA), 1,4,7-triazacyclononane-N,N′,N″-triacetic acid (NOTA), ethylenediaminetetraacetic acid (EDTA), diethylenetriaminepentaacetic acid (DTPA), 1,4,7-triazacyclononane-N,N′,N″-triacetic acid (NOTA), ({4-[2-(bis-carboxymethyl-amino)-ethyl]-7-carboxymethyl-[1,4,7]triazonan-1-yl}acetic acid (NETA), and p-bromoacetamido-benzyl-tetraethylaminetetraacetic acid (TETA).

In one embodiment, the theranostic agent comprises a compound having the chemical formula:

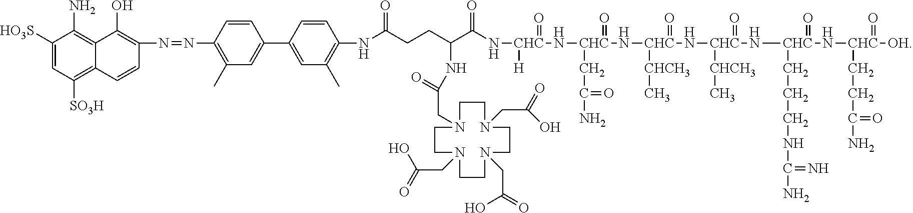

In certain embodiments, the theranostic agent further comprises a detectable label. In some embodiments, the detectable label is a diagnostic metal ion that is chelated by the chelating agent. In other embodiments, the compound is labeled with a radionuclide such as a radiohalogen.

In certain embodiments, the diagnostic metal ion is a metal radionuclide detectable by positron emission tomography (PET) or single photon emission computed tomography (SPECT). Exemplary metal radionuclides that can be used include, without limitation, ⁶⁴Cu, ⁶⁷Cu, ⁸⁹Zr, ⁸⁶Y, ¹¹¹In, ¹⁷⁷Lu, ²⁰¹Tl, ^{99m}Tc, ⁶⁷Ga, ⁶⁸Ga, ⁴⁴Sc, and ⁸²Rb.

In certain embodiments, the compound is labeled with a radiohalogen detectable by PET or SPECT. Exemplary radiohalogens that can be used include, without limitation, ¹⁸F, ¹³¹I, ¹²⁵I, ¹²⁴I, and ¹²³I.

In certain embodiments, the therapeutic radionuclide is a metal radionuclide or a radiohalogen such as a beta-emitter or alpha-emitter suitable for radiotherapy. Exemplary radionuclides that can be used include, without limitation, ⁹⁰Y, ¹⁷⁷Lu, ⁶⁷Cu, ¹³¹I, ²¹¹At, ²¹³Bi, ²¹²Pb, and ²²⁵AC.

In certain embodiments, the diagnostic metal ion is a paramagnetic metal ion detectable by magnetic resonance imaging (MRI). Exemplary paramagnetic metal ions that can be used include, without limitation, Mn²⁺, Fe³⁺, Fe²⁺, and Gd³⁺.

In certain embodiments, the theranostic agent further comprises an anti-cancer therapeutic agent. In some embodiments, the anti-cancer therapeutic agent is conjugated to the TMTP1 peptide. Fusing anti-cancer therapeutic agents to the theranostic agent can increase the anti-cancer efficacy of the theranostic against metastatic cells. Exemplary anti-cancer therapeutic agents include, without limitation, toxins, radioisotopes, immunomodulators, angiogenesis inhibitors, therapeutic enzymes, and cytotoxic or pro-apoptotic agents for treatment of cancer.

In another aspect, a composition is provided comprising a theranostic agent, described herein, and a pharmaceutically acceptable excipient. In some embodiments, the composition further comprises an anti-cancer therapeutic agent. Exemplary anti-cancer therapeutic agents include, without limitation, chemotherapeutic agents, immunotherapeutic agents, biologic therapeutic agents, pro-apoptotic agents, angiogenesis inhibitors, photoactive agents, radiosensitizing drugs, and radioisotopes.

In another aspect, a kit is provided comprising a composition comprising a theranostic agent and instructions for using the kit for detecting and treating metastatic cancer as described herein.

In another aspect, a method of imaging cancerous tissue of a patient suspected of having metastatic cancer is provided, the method comprising: a) contacting tissue of the patient suspected of being cancerous with a detectably effective amount of the theranostic agent under conditions wherein metastatic cancerous cells, if present, in the tissue uptake the theranostic agent; and b) imaging the tissue of the patient, wherein detection of increased uptake of the theranostic agent into the tissue of the patient compared to a control indicates that the patient has metastatic cancer.

In certain embodiments, the tissue is contacted with the theranostic agent in vivo or in vitro.

In certain embodiments, imaging is performed using positron emission tomography (PET), single photon emission computed tomography (SPECT), magnetic resonance imaging (MRI), positron emission tomography-computed tomography (PET-CT), positron emission tomography-magnetic resonance imaging (PET-MRI), or single photon emission computed tomography-magnetic resonance imaging (SPECT-MRI).

In another aspect, a method of monitoring progression of metastatic cancer in a patient is provided, the method comprising: imaging tissue of the patient with a theranostic agent according to the methods described herein, wherein a first image is obtained at a first time point and a second image is obtained later at a second time point, wherein detection of increased uptake of the theranostic agent into the tissue of the patient at the second time point compared to the first time point indicates that the patient is worsening, and detection of decreased uptake of the theranostic agent into the tissue of the patient at the second time point compared to the first time point indicates that the patient is improving. For example, increased uptake of the theranostic agent into the tissue of the patient may be associated with growth of a tumor or the presence of more tumors or more metastatic cancerous cells at the second time point, which can be determined by inspection of the images. Alternatively, decreased uptake of the theranostic agent into the tissue of the patient may be caused, for example, by tumor shrinkage or the presence of fewer tumors or fewer metastatic cancerous cells.

In another aspect, a method for evaluating the effect of an agent for treating cancer in a patient is provided, the method comprising: imaging tissue of the patient with a theranostic agent according to the methods described herein before and after the patient is treated with the agent, wherein detection of increased uptake of the theranostic agent into the tissue of the patient (e.g., from tumor growth or increase in number of tumors or metastatic cancerous cells) after the patient is treated with said agent compared to before the patient is treated with the agent indicates that the patient is worsening, and decreased uptake of the theranostic agent into the tissue of the patient (e.g., from reduction in tumor size or reduction in the number of metastatic cancerous cells) after the subject is treated with the agent compared to before the patient is treated with the agent indicates that the patient is improving.

In another aspect, a method for treating cancer is provided, the method comprising administering to a subject in need thereof a therapeutically effective amount of a composition comprising a theranostic agent comprising an anti-cancer agent, as described herein. Multiple cycles of treatment may be administered to a subject. In certain embodiments, the theranostic agent is administered according to a daily dosing regimen or intermittently. Preferably, the theranostic agent is administered for a time period sufficient to effect at least a partial tumor response, and more preferably a complete tumor response in the subject.

A theranostic agent may be administered by any suitable mode of administration. In certain embodiments, the theranostic agent is administered intravenously, subcutaneously, or intralesionally to a subject. In another embodiment, the theranostic agent is administered locally at a site of a tumor or cancerous cells in the subject.

In another embodiment, the method further comprises performing surgery, radiation therapy, chemotherapy, immunotherapy, or biologic therapy, or a combination thereof.

In certain embodiments, the method further comprises monitoring uptake of the theranostic agent into metastatic cancerous cells of the subject by detecting the theranostic agent.

In certain embodiments, the method of further comprises recording one or more images of metastatic cancerous cells that uptake the theranostic agent in the subject.

In certain embodiments, the method further comprises monitoring anti-tumor activity of the theranostic agent by recording one or more images of the metastatic cancerous cells that uptake the theranostic agent in the subject.

In certain embodiments, one or more images are obtained using positron emission tomography (PET), positron emission tomography-computed tomography (PET-CT), positron emission tomography-magnetic resonance imaging (PET-MRI), or single photon emission computed tomography (SPECT).

In certain embodiments, the method further comprises administering an anti-cancer therapeutic agent. Exemplary anti-cancer therapeutic agents include, without limitation, a chemotherapeutic agent, an immunotherapeutic agent, a biologic therapeutic agent, a pro-apoptotic agent, an angiogenesis inhibitor, a photoactive agent, radiosensitizing drugs, and a radioisotope.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1 shows the structure of DOTA-EB-TMTP1.

FIG. 2 shows the analytical HPLC for [⁶⁴Cu]DOTA-EB-TMTP1, RAD signals were expressed as millivolt (mV). The chemical purity of [⁶⁴Cu]DOTA-EB-TMTP1 was >95% based on analytical HPLC.

FIG. 3 shows cell uptake of [⁶⁴Cu]DOTA-EB-TMTP1 (n=4, mean±SD); the uptake ratio was increased with time and reached the peak at 45 min.

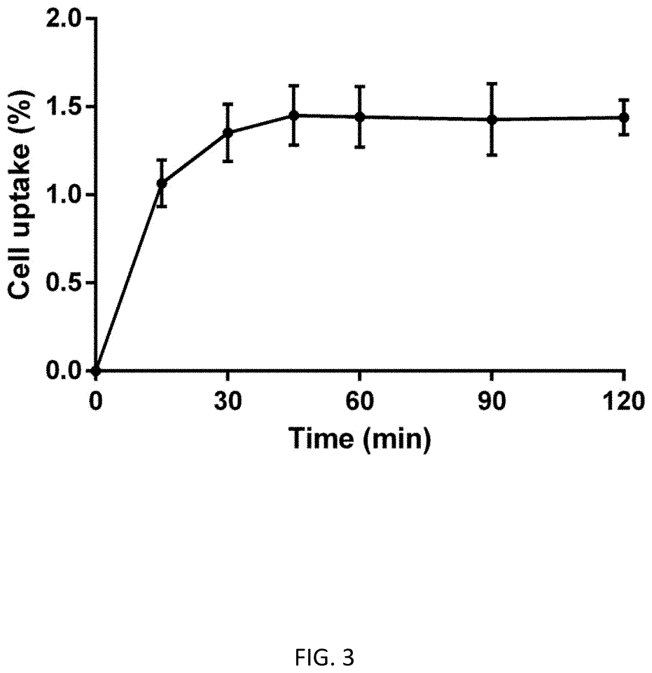

FIG. 4 shows inhibition of [⁶⁴Cu]DOTA-EB-TMTP1 binding to 143B cells by TMTP1, IC50=2.106e-007.

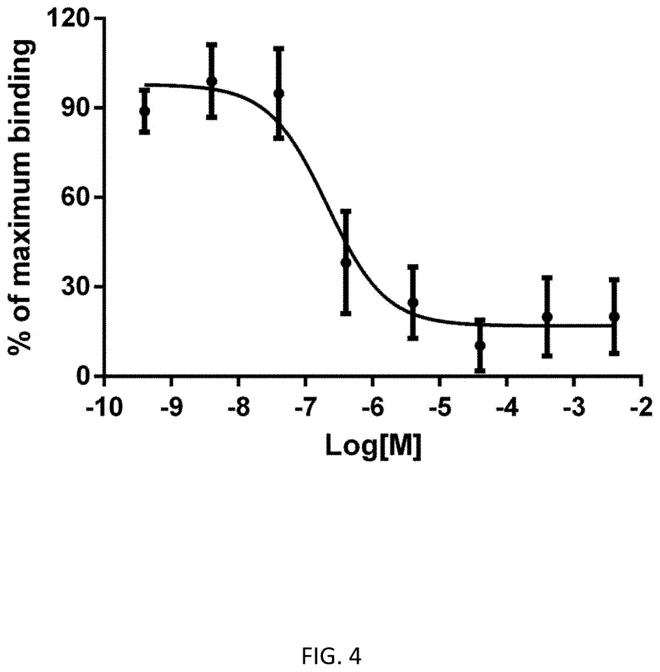

FIG. 5 shows our previous report that [¹⁸F]AIF-NOTA-G-TMTP1 specifically targets highly metastatic and/or aggressive hepatocellular carcinoma with low liver uptake (Li Y, Zhang D, Shi Y, et al. Contrast Media Mol Imaging 11:262-271).

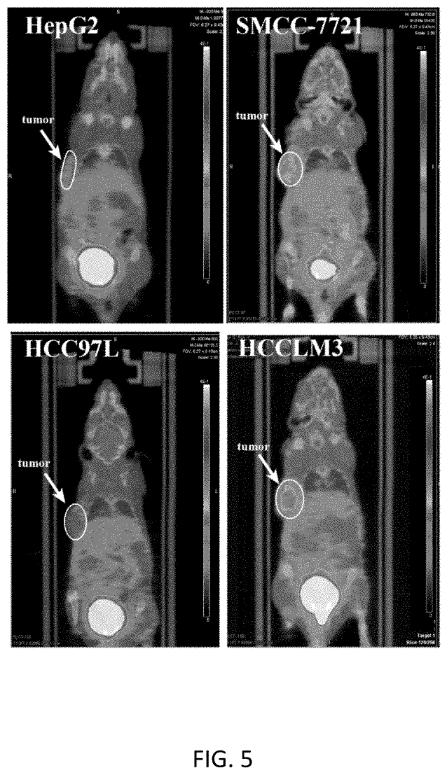

FIG. 6 shows representative whole-body coronal microPET/CT images of 143B tumor bearing nude mice model, which were acquired at 1, 2, 4, 8, 12, 36 and 48 hours after intravenous (i.v.) injection of 3.7-7.4 MBq (100-200 μCO of [⁶⁴Cu]DOTA-EB-TMTP1. Tumors were indicated in while circles.

FIG. 7 shows representative time activity curves of [⁶⁴Cu]DOTA-EB-TMTP1 tissue uptake by heart, tumor, muscle and kidney in 143B cell lines tumor bearing nude mice model. The tumor uptake of [⁶⁴Cu]DOTA-EB-TMTP1 was increased as time goes by, and reached the plateau of 6.5±0.8% ID/g at 8 h after injection.

FIG. 8 shows biodistribution of [⁶⁴Cu]DOTA-EB-TMTP1 at 48 h after injection in 143B tumor bearing nude mice. Tumor uptake was 6.7±2 0.9% ID/g.

### DETAILED DESCRIPTION OF EMBODIMENTS

Theranostic agents useful for imaging and treating metastatic cancer are provided. The theranostic agents comprise a TMTP1 peptide conjugated to an albumin binding moiety to prolong circulation lifetime and a chelating agent to allow complexation of a diagnostic metal ion with the theranostic agent. The theranostic agent can be conjugated, for example, to a positron-emitting metal radionuclide suitable for PET imaging such as 64Cu, ⁶⁸Ga, ⁴⁴Sc, ⁸⁶Y, ⁸⁹Zr, or ⁸²Rb; or a gamma-emitting metal radionuclide suitable for SPECT imaging such as 67Ga, ⁹⁹mTc, ¹¹¹In, or ¹⁷⁷Lu. Alternatively, theranostic agents can be conjugated with a paramagnetic metal ion suitable for use in magnetic resonance imaging (MRI) such as manganese (e.g., Mn²⁺), iron (e.g., Fe³⁺, Fe²⁺) or gadolinium (e.g., Gd³⁺). The theranostic agent may also be labeled with a positron-emitting radiohalogen such as 124I or ¹⁸F suitable for PET imaging or a gamma-emitting radiohalogen such as ¹³¹I, ¹²⁵I, or ¹²³I suitable for SPECT imaging. In addition, the theranostic agent can be conjugated with radionuclides suitable for radiotherapy such as ¹⁷⁷Lu, ⁹⁰Y, ²¹³Bi, ²¹²Pb, ²¹¹At, ²²⁵Ac, ¹⁶⁶Ho, ⁸⁹Sr, ¹⁵³Sm, ²²³Ra, ²²⁶Ra, ¹³⁷Cs, ¹⁹⁸Au, ¹⁸²Ta, ¹⁹²Ir, ¹²⁵I, or ¹³¹I. Such theranostic agents show selective binding to metastatic cancer cells and accumulation in metastatic tumors, and are useful for PET, SPECT, or MRI imaging and radionuclide treatment of metastatic cells in vitro and in vivo.

Before the theranostic agents and methods of imaging and treating metastatic cancer are described, it is to be understood that this invention is not limited to a particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a theranostic agent” includes a plurality of such theranostic agents and reference to “the cancerous cell” includes reference to one or more cancerous cells and equivalents thereof, such as cancer cells, tumor cells, neoplastic cells, and malignant cells, known to those skilled in the art, and so forth.

By “isolated” is meant, when referring to a polypeptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

The term “conjugated” refers to the joining by covalent or noncovalent means of two compounds or agents (e.g., theranostic agent conjugated to a diagnostic metal ion suitable for PET, SPECT, or MRI imaging, or a radioisotope suitable for radiotherapy).

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted (e.g., those with cancer) as well as those in which prevention is desired (e.g., those with increased susceptibility to cancer, those suspected of having cancer, those with a risk of recurrence, etc.).

The terms “tumor,” “cancer” and “neoplasia” are used interchangeably and refer to a cell or population of cells whose growth, proliferation or survival is greater than growth, proliferation or survival of a normal counterpart cell, e.g. a cell proliferative, hyperproliferative or differentiative disorder. Typically, the growth is uncontrolled. The term “malignancy” refers to invasion of nearby tissue. The term “metastasis” or a secondary, recurring or recurrent tumor, cancer or neoplasia refers to spread or dissemination of a tumor, cancer or neoplasia to other sites, locations or regions within the subject, in which the sites, locations or regions are distinct from the primary tumor or cancer. Neoplasia, tumors and cancers include benign, malignant, metastatic and non-metastatic types, and include any stage (I, II, III, IV or V) or grade (G1, G2, G3, etc.) of neoplasia, tumor, or cancer, or a neoplasia, tumor, cancer or metastasis that is progressing, worsening, stabilized or in remission. In particular, the terms “tumor,” “cancer” and “neoplasia” include carcinomas, such as squamous cell carcinoma, adenocarcinoma, adenosquamous carcinoma, anaplastic carcinoma, large cell carcinoma, and small cell carcinoma, and include cancers such as, but are not limited to, head and neck cancer, skin cancer, breast cancer, ovarian cancer, melanoma, pancreatic cancer, peripheral neuroma, glioblastoma, adrenocortical carcinoma, AIDS-related lymphoma, anal cancer, bladder cancer, meningioma, glioma, astrocytoma, cervical cancer, chronic myeloproliferative disorders, colon cancer, endometrial cancer, ependymoma, esophageal cancer, Ewing's sarcoma, extracranial germ cell tumors, extrahepatic bile duct cancer, gallbladder cancer, gastric cancer, gastrointestinal carcinoid tumors, gestational trophoblastic tumors, hairy cell leukemia, Hodgkin lymphoma, non-Hodgkin lymphoma, hypopharyngeal cancer, islet cell carcinoma, Kaposi sarcoma, laryngeal cancer, leukemia, lip cancer, oral cavity cancer, liver cancer, male breast cancer, malignant mesothelioma, medulloblastoma, Merkel cell carcinoma, metastatic squamous neck cell carcinoma, multiple myeloma and other plasma cell neoplasms, mycosis fungoides and the Sezary syndrome, myelodysplastic syndromes, nasopharyngeal cancer, neuroblastoma, non-small cell lung cancer, small cell lung cancer, oropharyngeal cancer, bone cancers, including osteosarcoma and malignant fibrous histiocytoma of bone, paranasal sinus cancer, parathyroid cancer, penile cancer, pheochromocytoma, pituitary tumors, prostate cancer, rectal cancer, renal cell cancer, retinoblastoma, rhabdomyosarcoma, salivary gland cancer, small intestine cancer, soft tissue sarcoma, supratentorial primitive neuroectodermal tumors, pineoblastoma, testicular cancer, thymoma, thymic carcinoma, thyroid cancer, transitional cell cancer of the renal pelvis and ureter, urethral cancer, uterine sarcoma, vaginal cancer, vulvar cancer, and Wilm's tumor and other childhood kidney tumors.

By “anti-tumor activity” is intended a reduction in the rate of cell proliferation, and hence a decline in growth rate of an existing tumor or in a tumor that arises during therapy, and/or destruction of existing neoplastic (tumor) cells or newly formed neoplastic cells, and hence a decrease in the overall size of a tumor during therapy. Such activity can be assessed using animal models.

By “therapeutically effective dose or amount” of a theranostic agent is intended an amount that, when administered as described herein, brings about a positive therapeutic response, such as anti-tumor activity. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein.

The term “tumor response” as used herein means a reduction or elimination of all measurable lesions. The criteria for tumor response are based on the WHO Reporting Criteria [WHO Offset Publication, 48-World Health Organization, Geneva, Switzerland, (1979)]. Ideally, all uni- or bidimensionally measurable lesions should be measured at each assessment. When multiple lesions are present in any organ, such measurements may not be possible and, under such circumstances, up to 6 representative lesions should be selected, if available.

The term “complete response” (CR) as used herein means a complete disappearance of all clinically detectable malignant disease, determined by 2 assessments at least 4 weeks apart.

The term “partial response” (PR) as used herein means a 50% or greater reduction from baseline in the sum of the products of the longest perpendicular diameters of all measurable disease without progression of evaluable disease and without evidence of any new lesions as determined by at least two consecutive assessments at least four weeks apart. Assessments should show a partial decrease in the size of lytic lesions, recalcifications of lytic lesions, or decreased density of blastic lesions.

“Substantially purified” generally refers to isolation of a substance (compound, drug, polynucleotide, protein, polypeptide) such that the substance comprises the majority percent of the sample in which it resides. Typically in a sample, a substantially purified component comprises 50%, preferably 80%-85%, more preferably 90-95% of the sample. Techniques for purifying substances of interest are well-known in the art and include, for example, ion-exchange chromatography, affinity chromatography and sedimentation according to density.

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. Preferably, the mammal is human.

As used herein, the terms “detectable label”, “detection agent”, “diagnostic agent”, and “detectable moiety” are used interchangeably and refer to a molecule or substance capable of detection, including, but not limited to, fluorescers, chemiluminescers, chromophores, bioluminescent proteins, enzymes, enzyme substrates, enzyme cofactors, enzyme inhibitors, isotopic labels, semiconductor nanoparticles, dyes, metal ions, metal sols, ligands (e.g., biotin, streptavidin or haptens) and the like. The term “fluorescer” refers to a substance or a portion thereof which is capable of exhibiting fluorescence in the detectable range. Particular examples of detectable labels which may be used in the practice of the invention include isotopic labels, including radioactive (e.g., alpha, beta, gamma, or positron-emitting radionuclides) and non-radioactive isotopes, such as, ³H, ²H, ¹²⁰I, ¹²³I, ¹²⁴I, ¹²⁵I, ¹³¹I, ³⁵S, ¹¹C, ¹³C, ¹⁴C, ³²P, ¹⁵N, ¹³N, ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹⁵⁴Gd, ¹⁵⁵Gd, ¹⁵⁶Gd, ¹⁵⁷Gd, ¹⁵⁸Gd, ¹⁵O, ¹⁸⁶Re, ¹⁸⁸Re, ⁵¹M, ^{52m}Mn, ⁵⁵Co, ⁷²As, ⁷⁵Br, ⁷⁶Br, ^{82m}Rb, and ⁸³Sr. In particular, detectable labels may comprise positron-emitting radionuclides suitable for PET imaging such as, but not limited to, ⁶⁴Cu, ⁸⁹Zr, ⁶⁸Ga, ¹⁷⁷Lu, ⁸²Rb, ¹¹O, ¹³N, ¹⁵O, and ¹⁸F; gamma-emitting radionuclides suitable for single photon emission computed tomography (SPECT) imaging such as, but not limited to, ⁶⁷Ga, ^{99m}Tc, ¹²³I, and ¹³¹I; and radionuclides suitable for radiotherapy such as ¹⁷⁷Lu, ⁹⁰Y, ²¹³Bi, ²¹²Pb, ²¹¹At, ²²⁵Ac, ¹⁶⁶Ho, ⁸⁹Sr, ¹⁵³Sm, ²²³Ra, ²²⁶Ra, ¹³⁷Cs, ¹⁹⁸Au, ¹⁸²Ta, ¹⁹²Ir, ¹²⁵I, or ¹³¹I. Detectable labels may also include non-radioactive, paramagnetic metal ions suitable for MRI imaging such as, but not limited to, Mn²⁺, Fe³⁺, Fe²⁺, Gd³⁺, Ti²⁺, Cr³⁺, Co²⁺, Ni²⁺, and Cu²⁺. Detectable labels may also include fluorophores including without limitation, SYBR green, SYBR gold, a CAL Fluor dye such as CAL Fluor Gold 540, CAL Fluor Orange 560, CAL Fluor Red 590, CAL Fluor Red 610, and CAL Fluor Red 635, a Quasar dye such as Quasar 570, Quasar 670, and Quasar 705, an Alexa Fluor such as Alexa Fluor 350, Alexa Fluor 488, Alexa Fluor 546, Alexa Fluor 555, Alexa Fluor 594, Alexa Fluor 647, and Alexa Fluor 784, a cyanine dye such as Cy 3, Cy3.5, Cy5, Cy5.5, and Cy7, fluorescein, 2′, 4′, 5′, 7′-tetrachloro-4-7-dichlorofluorescein (TET), carboxyfluorescein (FAM), 6-carboxy-4′,5′-dichloro-2′,7′-dimethoxyfluorescein (JOE), hexachlorofluorescein (HEX), rhodamine, carboxy-X-rhodamine (ROX), tetramethyl rhodamine (TAMRA), FITC, dansyl, umbelliferone, dimethyl acridinium ester (DMAE), Texas red, luminol, and quantum dots, enzymes such as alkaline phosphatase (AP), beta-lactamase, chloramphenicol acetyltransferase (CAT), adenosine deaminase (ADA), aminoglycoside phosphotransferase (neo^{r}, G418^{r}) dihydrofolate reductase (DHFR), hygromycin-B-phosphotransferase (HPH), thymidine kinase (TK), β-galactosidase (lacZ), and xanthine guanine phosphoribosyltransferase (XGPRT), beta-glucuronidase (gus), placental alkaline phosphatase (PLAP), and secreted embryonic alkaline phosphatase (SEAP). Enzyme tags are used with their cognate substrate. The terms also include chemiluminescent labels such as luminol, isoluminol, acridinium esters, and peroxyoxalate and bioluminescent proteins such as firefly luciferase, bacterial luciferase, Renilla luciferase, and aequorin. The terms also include color-coded microspheres of known fluorescent light intensities (see e.g., microspheres with xMAP technology produced by Luminex (Austin, Tex.); microspheres containing quantum dot nanocrystals, for example, containing different ratios and combinations of quantum dot colors (e.g., Qdot nanocrystals produced by Life Technologies (Carlsbad, Calif.); glass coated metal nanoparticles (see e.g., SERS nanotags produced by Nanoplex Technologies, Inc. (Mountain View, Calif.); barcode materials (see e.g., sub-micron sized striped metallic rods such as Nanobarcodes produced by Nanoplex Technologies, Inc.), encoded microparticles with colored bar codes (see e.g., CellCard produced by Vitra Bioscience, vitrabio.com), glass microparticles with digital holographic code images (see e.g., CyVera microbeads produced by Illumina (San Diego, Calif.), near infrared (NIR) probes, and nanoshells. The terms also include contrast agents such as ultrasound contrast agents (e.g. SonoVue microbubbles comprising sulfur hexafluoride, Optison microbubbles comprising an albumin shell and octafluoropropane gas core, Levovist microbubbles comprising a lipid/galactose shell and an air core, Perflexane lipid microspheres comprising perfluorocarbon microbubbles, and Perflutren lipid microspheres comprising octafluoropropane encapsulated in an outer lipid shell), magnetic resonance imaging (MRI) contrast agents (e.g., gadodiamide, gadobenic acid, gadopentetic acid, gadoteridol, gadofosveset, gadoversetamide, gadoxetic acid), and radiocontrast agents, such as for computed tomography (CT), radiography, or fluoroscopy (e.g., diatrizoic acid, metrizoic acid, iodamide, iotalamic acid, ioxitalamic acid, ioglicic acid, acetrizoic acid, iocarmic acid, methiodal, diodone, metrizamide, iohexol, ioxaglic acid, iopamidol, iopromide, iotrolan, ioversol, iopentol, iodixanol, iomeprol, iobitridol, ioxilan, iodoxamic acid, iotroxic acid, ioglycamic acid, adipiodone, iobenzamic acid, iopanoic acid, iocetamic acid, sodium iopodate, tyropanoic acid, and calcium iopodate). The detectable label may be attached indirectly or directly to a theranostic agent described herein, wherein the label facilitates the detection of the theranostic agent in which it is incorporated.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly salts containing pharmaceutically acceptable cations include, but are not limited to, sodium, potassium, calcium, aluminum, lithium, and ammonium (including substituted ammonium). The term “antibody” encompasses monoclonal antibodies as well as hybrid antibodies, altered antibodies, chimeric antibodies, and humanized antibodies.

The term antibody includes: hybrid (chimeric) antibody molecules (see, for example, Winter et al. (1991) Nature 349:293-299; and U.S. Pat. No. 4,816,567); F(ab′)₂ and F(ab) fragments; Fᵥ molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (scFv) (see, e.g., Huston et al. (1988) Proc Natl Acad Sci USA 85:5879-5883); nanobodies or single-domain antibodies (sdAb) (see, e.g., Wang et al. (2016) Int J Nanomedicine 11:3287-3303, Vincke et al. (2012) Methods Mol Biol 911:15-26; dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Theranostic Agents

Theranostic agents useful for imaging and treating metastatic cancer are provided. The theranostic agents comprise a TMTP1 peptide (NVVRQ, SEQ ID NO:1) conjugated to an albumin binding moiety and a chelating agent. The TMTP1 peptide provides the theranostic agent with high affinity for metastatic tumor cells; the albumin binding moiety increases the blood circulation half-life of the theranostic agent; and the chelating agent allows complexation of the theranostic agent with a radioactive metal, paramagnetic ion, or other diagnostic cation. Such theranostic agents show selective uptake by metastatic cancer cells and are useful for PET, SPECT, or MRI imaging and radionuclide treatment of metastatic cells in vitro or in vivo.

In certain embodiments, the albumin binding moiety is an Evans blue dye (tetrasodium (6E,6′E)-6,6-[(3,3′-dimethylbiphenyl-4,4′-diyl)di(1E) hydrazin-2-yl-1-ylidene]bis(4-amino-5-oxo-5,6-dihydronaphthalene-1,3-disulfonate)), or an Evans blue derivative such as a truncated Evans blue (tEB) dye, wherein a 1-amino-naphthol-2,4-disulfonic acid moiety of Evans blue is replaced with the chelating agent to allow metal complexation to the theranostic agent. The inclusion of the Evans blue or a truncated derivative thereof also increases uptake of the theranostic agent by metastatic cancerous cells. Alternatively, fatty acids (e.g., palmitic acid, myristic acid), biliary acids, small molecules (e.g., diphenylcyclohexane, iopanoic acid, 2,4,6-triiodobenzoic acid), and antibodies having affinity for albumin may also be used to prolong circulation time of the theranostic agent.

Chelating agents can be coupled to the TMTP1 peptide in the theranostic agent to confer metal binding capability. Exemplary chelating agents include, without limitation, 1,4,7,10-tetraazacyclododecane-1,4,7,10-tetraacetic acid (DOTA), 1,4,7-triazacyclononane-N,N′,N″-triacetic acid (NOTA), ethylenediaminetetraacetic acid (EDTA), diethylenetriaminepentaacetic acid (DTPA), 1,4,7-triazacyclononane-N,N′,N″-triacetic acid (NOTA), ({4-[2-(bis-carboxymethyl-amino)-ethyl]-7-carboxymethyl-[1,4,7]triazonan-1-yl}acetic acid (NETA), and p-bromoacetamido-benzyl-tetraethylaminetetraacetic acid (TETA), porphyrins, polyamines, crown ethers, bis-thiosemicarbazones, polyoximes, and like groups known to be useful for this purpose.

In some embodiments, the chelating agent of the theranostic agent is used to bind a radioactive metal, paramagnetic ion, or other diagnostic cation. Particular examples of cations that may be chelated by the theranostic agent include metal radionuclides detectable by positron emission tomography (PET) or single photon emission computed tomography (SPECT). For example, the theranostic agent can be conjugated to a detectable label comprising a positron-emitting metal radionuclide suitable for PET imaging such as ⁶⁴Cu, ⁶⁸Ga, ⁴⁴Sc, ⁸⁶Y, ⁸⁹Zr, or ⁸²Rb; or a gamma-emitting metal radionuclide suitable for single photon emission computed tomography (SPECT) imaging such as ⁶⁷Ga, ^{99m}Tc, ¹¹¹In, or ¹⁷⁷Lu. The theranostic agent may also be labeled with a positron-emitting radiohalogen such as ¹²⁴I or ¹⁸F suitable for PET imaging or a gamma-emitting radiohalogen such as ¹³¹I, ¹²⁵I, or ¹²³I suitable for SPECT imaging. Such theranostic agents show selective uptake by metastatic cancer cells and are useful for PET or SPECT imaging and/or treatment of metastatic cells in vitro and in vivo. The same chelating agents, when complexed with paramagnetic metal ions are useful for MRI. For example, theranostic agents can be complexed with paramagnetic metal ions, including, without limitation, those of manganese (e.g., Mn²⁺), iron (e.g., Fe³⁺, Fe²⁺) and gadolinium (e.g., Gd³⁺) for use as contrast agents in MRI. In addition, the theranostic agent can be conjugated with radionuclides suitable for radiotherapy such as ¹⁷⁷Lu, ⁹⁰Y, ²¹³a, ²¹²Pb, ²¹¹At, ²²⁵Ac, ¹⁶⁶Ho, ⁸⁹Sr, ¹⁵³Sm, ²²³Ra, ²²⁶Ra, ¹³⁷Cs, ¹⁹⁸Au, ¹⁸²Ta, ¹⁹²Ir, ¹²⁵I, or ¹³¹I.

In one embodiment, the theranostic agent comprises a compound comprising a TMTP1 peptide conjugated to Evans blue (tEB) and DOTA (DOTA-tEB-TMTP1) having the chemical formula:

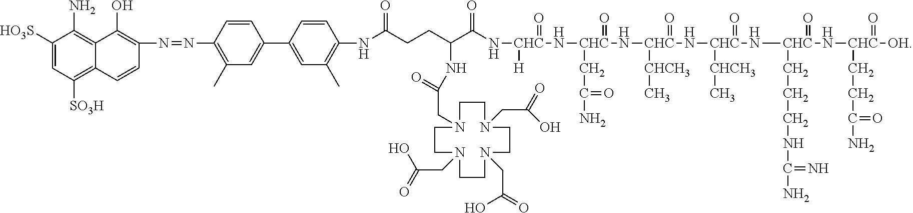

### Pharmaceutical Compositions

Theranostic agents can be formulated into pharmaceutical compositions optionally comprising one or more pharmaceutically acceptable excipients. Exemplary excipients include, without limitation, carbohydrates, inorganic salts, antimicrobial agents, antioxidants, surfactants, buffers, acids, bases, and combinations thereof. Excipients suitable for injectable compositions include water, alcohols, polyols, glycerine, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

A composition can also include an antimicrobial agent for preventing or deterring microbial growth. Nonlimiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the composition as well. Antioxidants are used to prevent oxidation, thereby preventing the deterioration of the theranostic agent or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant can be present as an excipient. Exemplary surfactants include: polysorbates, such as “Tween 20” and “Tween 80,” and pluronics such as F68 and F88 (BASF, Mount Olive, N.J.); sorbitan esters; lipids, such as phospholipids such as lecithin and other phosphatidylcholines, phosphatidylethanolamines (although preferably not in liposomal form), fatty acids and fatty esters; steroids, such as cholesterol; chelating agents, such as EDTA; and zinc and other such suitable cations.

Acids or bases can be present as an excipient in the composition. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of the theranostic agent (e.g., when contained in a drug delivery system) in the composition will vary depending on a number of factors, but will optimally be a therapeutically effective dose when the composition is in a unit dosage form or container (e.g., a vial). A therapeutically effective dose can be determined experimentally by repeated administration of increasing amounts of the composition in order to determine which amount produces a clinically desired endpoint.

The amount of any individual excipient in the composition will vary depending on the nature and function of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects. Generally, however, the excipient(s) will be present in the composition in an amount of about 1% to about 99% by weight, preferably from about 5% to about 98% by weight, more preferably from about 15 to about 95% by weight of the excipient, with concentrations less than 30% by weight most preferred. These foregoing pharmaceutical excipients along with other excipients are described in “Remington: The Science & Practice of Pharmacy”, 19th ed., Williams & Williams, (1995), the “Physician's Desk Reference”, 52nd ed., Medical Economics, Montvale, N.J. (1998), and Kibbe, A. H., Handbook of Pharmaceutical Excipients, 3rd Edition, American Pharmaceutical Association, Washington, D.C., 2000.

The compositions encompass all types of formulations and in particular those that are suited for injection, e.g., powders or lyophilates that can be reconstituted with a solvent prior to use, as well as ready for injection solutions or suspensions, dry insoluble compositions for combination with a vehicle prior to use, and emulsions and liquid concentrates for dilution prior to administration. Examples of suitable diluents for reconstituting solid compositions prior to injection include bacteriostatic water for injection, dextrose 5% in water, phosphate buffered saline, Ringer's solution, saline, sterile water, deionized water, and combinations thereof. With respect to liquid pharmaceutical compositions, solutions and suspensions are envisioned. Additional preferred compositions include those for oral, ocular, or localized delivery.

The pharmaceutical preparations herein can also be housed in a syringe, an implantation device, or the like, depending upon the intended mode of delivery and use. Preferably, the compositions comprising a theranostic agent described herein are in unit dosage form, meaning an amount of a conjugate or composition of the invention appropriate for a single dose, in a premeasured or pre-packaged form.

The compositions herein may optionally include one or more additional agents, such as drugs for treating cancer or other medications used to treat a subject for a condition or disease. Compounded preparations may include a theranostic agent and one or more drugs for treating cancer, such as, but not limited to, chemotherapeutic agents, immunotherapeutic agents, biologic therapeutic agents, pro-apoptotic agents, angiogenesis inhibitors, photoactive agents, radiosensitizing drugs, and radioisotopes. Alternatively, such agents can be contained in a separate composition from the composition comprising a theranostic agent and co-administered concurrently, before, or after the composition comprising the theranostic agent.

### Imaging

Preferably, a detectably effective amount of a theranostic agent is administered to a subject; that is, an amount that is sufficient to yield an acceptable image using the imaging equipment that is available for clinical use. A detectably effective amount of the theranostic agent may be administered in more than one injection if needed. The detectably effective amount of the theranostic agent needed for an individual may vary according to factors such as the degree of uptake of the theranostic agent into cancerous tissue, the age, sex, and weight of the individual, and the particular medical imaging method used. Optimization of such factors is within the level of skill in the art.

Imaging with theranostic agents can be used in assessing efficacy of therapeutic drugs in treating cancer. For example, images can be acquired after treatment with an anti-cancer therapy to determine if the individual is responding to treatment. In a subject with cancer, imaging with a theranostic agent can be used to evaluate whether a tumor is shrinking or growing. Further, the extent of cancerous disease (how far and where the cancer has spread) can be determined to aid in determining prognosis and evaluating optimal strategies for treatment (e.g., surgery, radiation, or chemotherapy).

Additionally, theranostic agents can be used in image-guided surgery. Tissue of interest suspected of containing cancerous cells or a tumor can be contacted with a theranostic agent, such that the theranostic agent accumulates in metastatic cancerous cells. Imaging of tissues labeled with the theranostic agent in this way can be used, for example, for detection of metastatic cells, tumor margin delineation, evaluation of the completeness of resection, and evaluation of the efficacy of treatment.

### Metastatic Cancer-Targeted Therapeutic Agents

The theranostic agents described herein localize specifically to metastatic cancerous cells (see Example 1). Thus, the theranostic agents can also be used to target therapeutic agents to the location of metastatic tumors or cancerous cells to directly treat cancer in a subject. A theranostic agent can be conjugated to one or more therapeutic, anti-cancer agents, such as, but not limited to, drugs, toxins, radioisotopes, immunomodulators, angiogenesis inhibitors, therapeutic enzymes, and cytotoxic or pro-apoptotic agents for treatment of cancer.

For example, a theranostic agent can be conjugated to one or more chemotherapeutic agents such as, but not limited to, abitrexate, adriamycin, adrucil, amsacrine, asparaginase, anthracyclines, azacitidine, azathioprine, bicnu, blenoxane, busulfan, bleomycin, camptosar, camptothecins, carboplatin, carmustine, cerubidine, chlorambucil, cisplatin, cladribine, cosmegen, cytarabine, cytosar, cyclophosphamide, cytoxan, dactinomycin, docetaxel, doxorubicin, daunorubicin, ellence, elspar, epirubicin, etoposide, fludarabine, fluorouracil, fludara, gemcitabine, gemzar, hycamtin, hydroxyurea, hydrea, idamycin, idarubicin, ifosfamide, ifex, irinotecan, lanvis, leukeran, leustatin, matulane, mechlorethamine, mercaptopurine, methotrexate, mitomycin, mitoxantrone, mithramycin, mutamycin, myleran, mylosar, navelbine, nipent, novantrone, oncovin, oxaliplatin, paclitaxel, paraplatin, pentostatin, platinol, plicamycin, procarbazine, purinethol, ralitrexed, taxotere, taxol, teniposide, thioguanine, tomudex, topotecan, valrubicin, velban, vepesid, vinblastine, vindesine, vincristine, vinorelbine, VP-16, and vumon.

Alternatively or additionally, a theranostic agent can be conjugated to, one or more tyrosine-kinase inhibitors, such as Imatinib mesylate (Gleevec, also known as STI-571), Gefitinib (Iressa, also known as ZD1839), Erlotinib (marketed as Tarceva), Sorafenib (Nexavar), Sunitinib (Sutent), Dasatinib (Sprycel), Lapatinib (Tykerb), Nilotinib (Tasigna), and Bortezomib (Velcade); Janus kinase inhibitors, such as tofacitinib; ALK inhibitors, such as crizotinib; Bcl-2 inhibitors, such as obatoclax and gossypol; PARP inhibitors, such as Iniparib and Olaparib; PI3K inhibitors, such as perifosine; VEGF Receptor 2 inhibitors, such as Apatinib; AN-152 (AEZS-108) doxorubicin linked to [D-Lys(6)]-LHRH; Braf inhibitors, such as vemurafenib, dabrafenib, and LGX818; MEK inhibitors, such as trametinib; CDK inhibitors, such as PD-0332991 and LEE011; Hsp90 inhibitors, such as salinomycin; and/or small molecule drug conjugates, such as Vintafolide; serine/threonine kinase inhibitors, such as Temsirolimus (Torisel), Everolimus (Afinitor), Vemurafenib (Zelboraf), Trametinib (Mekinist), and Dabrafenib (Tafinlar).

In another example, a theranostic agent is conjugated to a hormonal blocking therapeutic agent for treatment of a cancer depending on estrogen for growth (e.g., cancer expressing estrogen receptors (ER+ cancer)). For example, the anti-B7-H3 antibody can be conjugated to a drug that blocks ER receptors (e.g. tamoxifen) or a drug that blocks the production of estrogen, such as an aromatase inhibitor (e.g. anastrozole, or letrozole).

In another example, the theranostic agent is conjugated to a toxin. The toxin can be of animal, plant or microbial origin. Exemplary toxins include Pseudomonas exotoxin, ricin, abrin, Staphylococcal enterotoxin-A, pokeweed antiviral protein, gelonin, diphtheria toxin, and Pseudomonas endotoxin.

In a further example, the theranostic agent is conjugated to an immunomodulator, such as a cytokine, a lymphokine, a monoline, a stem cell growth factor, a lymphotoxin (LT), a hematopoietic factor, a colony stimulating factor (CSF), an interferon (IFN), parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, a transforming growth factor (TGF), such as TGF-α or TGF-β, insulin-like growth factor (IGF), erythropoietin, thrombopoietin, a tumor necrosis factor (TNF) such as TNF-α or TNF-β, a mullerian-inhibiting substance, mouse gonadotropin-associated peptide, inhibin, activin, vascular endothelial growth factor, integrin, granulocyte-colony stimulating factor (G-CSF), granulocyte macrophage-colony stimulating factor (GM-CSF), an interferon such as interferon-α, interferon-β, or interferon-γ, S1 factor, an interleukin (IL) such as IL-1, IL-1cc, IL-2, IL-3, IL-4, IL-5, IL-6, IL-7, IL-8, IL-9, IL-10, IL-11, IL-12, IL-13, IL-14, IL-15, IL-16, IL-17, IL-18 IL-21 or IL-25, LIF, kit-ligand, FLT-3, angiostatin, thrombospondin, endostatin, and LT.

In another embodiment, the theranostic agent is conjugated to a radioactive isotope. Particularly useful therapeutic radionuclides include, but are not limited to ¹¹¹In, ¹⁷⁷Lu, ²¹¹At, ²¹²Pb, ²¹²Bi, ²¹³Bi, ²¹¹At, ⁶²Cu, ⁶⁴Cu, ⁶⁷Cu, ⁹⁰Y, ¹²³I, ¹²⁵I, ¹³¹I, ¹³⁷Cs, ³²P, ³³P, ⁷⁷Br, ⁴⁷SC, ¹¹¹Ag, ⁶⁷Ga, ¹⁴²Pr, ¹⁵³Sm, ¹⁶¹Tb, ¹⁶⁶Dy, ¹⁶⁶Ho, ¹⁸⁶Re, ¹⁸⁸Re, ¹⁸⁹Re, ²¹²Pb, ²²³Ra, ²²⁶Ra, ²²⁵Ac, ⁵⁹Fe, ⁷⁵Se, ⁷⁷As, ⁸⁹Sr, ⁹⁹Mo, ¹⁰⁵Rh, ¹⁰⁹Pd, ¹⁴³Pr, ¹⁴⁹Pm, ¹⁶⁹Er, ¹⁸²Ta, ¹⁹²Ir, ¹⁹⁴Ir, ¹⁹⁸Au, and ¹⁹⁹Au.

In certain embodiments, the therapeutic radionuclide has a decay energy in the range of 20 to 6,000 keV (e.g., 60 to 200 keV for an Auger emitter, 100-2,500 keV for a beta emitter, and 4,000-6,000 keV for an alpha emitter). In one embodiment, the radionuclide is an Auger-emitter (e.g., Co-58, Ga-67, Br-80m, Tc-99m, Rh-103m, Pt-109, In-111, Sb-119, 1-125, Ho-161, Os-189m and Ir-192). In another embodiment, the radionuclide is an alpha-emitter (e.g., Dy-152, At-211, Bi-212, Ra-223, Rn-219, Po-215, Bi-211, Ac-225, Fr-221, At-217, Bi-213 and Fm-255).

Additional therapeutic radioisotopes include ¹¹C, ¹³N, ¹⁵O, ⁷⁵Br, ¹⁹⁸Au, ²²⁴Ac, ¹²⁶I, ¹³³I, ⁷⁷Br, ^{113m}In, ⁹⁵Ru, ⁹⁷Ru, ¹⁰³Ru, ¹⁰⁵Ru, ¹⁰⁷Hg, ²⁰³Hg, ^{121m}Te, ^{122m}Te, ¹⁶⁵Tm, ¹⁶⁷Tm, ¹⁶⁸Tm, ¹⁹⁷Pt, ¹⁰⁹Pd, ¹⁰⁵Rh, ¹⁴²Pr, ¹⁴³Pr, ¹⁶¹Tb, ¹⁶⁶Ho, ¹⁹⁹Au, ⁵⁷Co, ⁵¹Cr, ⁵⁹Fe, ⁷⁵Se, ²⁰¹Tl, ²²⁵Ac, ⁷⁶Br, ¹⁶⁹Yb, and the like.

Theranostic agents may also be conjugated to a boron addend-loaded carrier for thermal neutron activation therapy. For example, boron addends such as carboranes, can be attached to B7-H3-targeting agents. Carboranes can be prepared with carboxyl functions on pendant side chains, as is well-known in the art. Attachment of carboranes to a carrier, such as aminodextran, can be achieved by activation of the carboxyl groups of the carboranes and condensation with amines on the carrier. The intermediate conjugate is then conjugated to the B7-H3-targeting agent. After administration of the B7-H3-targeting agent conjugate, a boron addend is activated by thermal neutron irradiation and converted to radioactive atoms which decay by alpha-emission to produce highly toxic, short-range effects.

### Administration

The methods described herein can be used for treating a subject for cancer. For example, theranostic agents can be used for treating a subject for a metastatic tumor or circulating metastatic tumor cells. At least one therapeutically effective cycle of treatment with a theranostic agent will be administered to a subject for treatment of cancer. By “therapeutically effective dose or amount” of a theranostic agent is intended an amount that when administered brings about a positive therapeutic response with respect to treatment of an individual for cancer. Of particular interest is an amount of a theranostic agent that provides an anti-tumor effect, as defined herein. By “positive therapeutic response” is intended the individual undergoing the treatment according to the invention exhibits an improvement in one or more symptoms of the cancer for which the individual is undergoing therapy.

Thus, for example, a “positive therapeutic response” would be an improvement in the disease in association with the therapy, and/or an improvement in one or more symptoms of the disease in association with the therapy. Therefore, for example, a positive therapeutic response would refer to one or more of the following improvements in the disease: (1) reduction in tumor size; (2) reduction in the number of metastatic cancer cells; (3) inhibition (i.e., slowing to some extent, preferably halting) of tumor growth; (4) inhibition (i.e., slowing to some extent, preferably halting) of cancer cell infiltration into peripheral organs; (5) inhibition (i.e., slowing to some extent, preferably halting) of tumor metastasis; and (6) some extent of relief from one or more symptoms associated with the cancer. Such therapeutic responses may be further characterized as to degree of improvement. Thus, for example, an improvement may be characterized as a complete response. By “complete response” is documentation of the disappearance of all symptoms and signs of all measurable or evaluable disease confirmed by physical examination, laboratory, ultrasound, nuclear, radiographic studies (i.e., CT (computer tomography), and/or MRI (magnetic resonance imaging)), and other non-invasive procedures repeated for all initial abnormalities or sites positive at the time of entry into the study. Alternatively, an improvement in the disease may be categorized as being a partial response. By “partial response” is intended a reduction of greater than 50% in the sum of the products of the perpendicular diameters of all measurable lesions when compared with pretreatment measurements.

In certain embodiments, multiple therapeutically effective doses of compositions comprising a theranostic agent and/or one or more other therapeutic agents, such as other drugs for treating cancer, or other medications will be administered according to a daily dosing regimen, or intermittently. For example, a therapeutically effective dose can be administered, one day a week, two days a week, three days a week, four days a week, or five days a week, and so forth. By “intermittent” administration is intended the therapeutically effective dose can be administered, for example, every other day, every two days, every three days, and so forth. For example, in some embodiments, a theranostic agent will be administered twice-weekly or thrice-weekly for an extended period of time, such as for 1, 2, 3, 4, 5, 6, 7, 8 . . . 10 . . . 15 . . . 24 weeks, and so forth. By “twice-weekly” or “two times per week” is intended that two therapeutically effective doses of the agent in question is administered to the subject within a 7-day period, beginning on day 1 of the first week of administration, with a minimum of 72 hours, between doses and a maximum of 96 hours between doses. By “thrice weekly” or “three times per week” is intended that three therapeutically effective doses are administered to the subject within a 7-day period, allowing for a minimum of 48 hours between doses and a maximum of 72 hours between doses. For purposes of the present invention, this type of dosing is referred to as “intermittent” therapy. In accordance with the methods of the present invention, a subject can receive intermittent therapy (i.e., twice-weekly or thrice-weekly administration of a therapeutically effective dose) for one or more weekly cycles until the desired therapeutic response is achieved. The agents can be administered by any acceptable route of administration as noted herein below.

The compositions comprising theranostic agents are typically, although not necessarily, administered orally, via injection (subcutaneously, intravenously, or intramuscularly), by infusion, or locally. Additional modes of administration are also contemplated, such as intra-arterial, intraperitoneal, pulmonary, nasal, topical, transdermal, intralesion, intrapleural, intraparenchymatous, rectal, transdermal, transmucosal, intrathecal, pericardial, intra-arterial, intraocular, and so forth. When administering the theranostic agent by injection, the administration may be by continuous infusion or by single or multiple boluses.

The preparations according to the invention are also suitable for local treatment. In a particular embodiment, a composition of the invention is used for localized delivery of a theranostic agent for the treatment of cancer. For example, compositions may be administered directly into a tumor or cancerous cells. Administration may be by perfusion through a regional catheter or direct intralesional injection.

The pharmaceutical preparation can be in the form of a liquid solution or suspension immediately prior to administration, but may also take another form such as a syrup, cream, ointment, tablet, capsule, powder, gel, matrix, suppository, or the like. The pharmaceutical compositions comprising a theranostic agent and other agents may be administered using the same or different routes of administration in accordance with any medically acceptable method known in the art.

In another embodiment, the pharmaceutical compositions comprising a theranostic agent and/or other agents are administered prophylactically, e.g., to prevent cancer progression or metastasis in tissue. Such prophylactic uses will be of particular value for subjects at high risk of cancer metastasis.

In another embodiment of the invention, the pharmaceutical compositions comprising a theranostic agent and/or other agents are in a sustained-release formulation, or a formulation that is administered using a sustained-release device. Such devices are well known in the art, and include, for example, transdermal patches, and miniature implantable pumps that can provide for drug delivery over time in a continuous, steady-state fashion at a variety of doses to achieve a sustained-release effect with a non-sustained-release pharmaceutical composition.

The invention also provides a method for administering a conjugate comprising a theranostic agent (e.g., conjugated to a diagnostic or therapeutic agent) as provided herein to a patient suffering from cancer. The method comprises administering, via any of the herein described modes, a therapeutically effective amount of the conjugate or drug delivery system, preferably provided as part of a pharmaceutical composition. The method of administering may be used to treat any cancer that is responsive to treatment with theranostic agent. More specifically, the compositions herein are effective in treating cancer.

Those of ordinary skill in the art will appreciate which conditions a theranostic agent can effectively treat. The actual dose to be administered will vary depending upon the age, weight, and general condition of the subject as well as the severity of the condition being treated, the judgment of the health care professional, and conjugate being administered. Therapeutically effective amounts can be determined by those skilled in the art, and will be adjusted to the particular requirements of each particular case.

Generally, a therapeutically effective amount will range from about 0.50 mg to 5 grams of a theranostic agent daily, more preferably from about 5 mg to 2 grams daily, even more preferably from about 7 mg to 1.5 grams daily. Preferably, such doses are in the range of 10-600 mg four times a day (QID), 200-500 mg QID, 25-600 mg three times a day (TID), 25-50 mg TID, 50-100 mg TID, 50-200 mg TID, 300-600 mg TID, 200-400 mg TID, 200-600 mg TID, 100 to 700 mg twice daily (BID), 100-600 mg BID, 200-500 mg BID, or 200-300 mg BID. The amount of compound administered will depend on the potency of the specific theranostic agent and the magnitude or effect desired and the route of administration.

A purified theranostic agent (again, preferably provided as part of a pharmaceutical preparation) can be administered alone or in combination with one or more other anti-cancer therapeutic agents, such as chemotherapy, immunotherapy, biologic or targeted therapy agents, or other medications used to treat a particular condition or disease according to a variety of dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, three times weekly, twice weekly, once weekly, twice monthly, once monthly, and any combination thereof. Preferred compositions are those requiring dosing no more than once a day.

A theranostic agent can be administered prior to, concurrent with, or subsequent to other agents. If provided at the same time as other agents, the theranostic agent can be provided in the same or in a different composition. Thus, the theranostic agent and other agents can be presented to the individual by way of concurrent therapy. By “concurrent therapy” is intended administration to a subject such that the therapeutic effect of the combination of the substances is caused in the subject undergoing therapy. For example, concurrent therapy may be achieved by administering a dose of a pharmaceutical composition comprising a theranostic agent and a dose of a pharmaceutical composition comprising at least one other agent, such as another drug for treating cancer, which in combination comprise a therapeutically effective dose, according to a particular dosing regimen. Similarly, the theranostic agent and one or more other therapeutic agents can be administered in at least one therapeutic dose. Administration of the separate pharmaceutical compositions can be performed simultaneously or at different times (i.e., sequentially, in either order, on the same day, or on different days), as long as the therapeutic effect of the combination of these substances is caused in the subject undergoing therapy.

Where a subject undergoing therapy in accordance with the previously mentioned dosing regimens exhibits a partial response or a relapse following a prolonged period of remission, subsequent courses of concurrent therapy may be needed to achieve complete remission of the disease. Thus, subsequent to a period of time off from a first treatment period, a subject may receive one or more additional treatment periods with theranostic agent. Such a period of time off between treatment periods is referred to herein as a time period of discontinuance. It is recognized that the length of the time period of discontinuance is dependent upon the degree of tumor response (i.e., complete versus partial) achieved with any prior treatment periods of concurrent therapy with these therapeutic agents.

Additionally, treatment with a theranostic agent may be combined with any other medical treatment for cancer, such as, but not limited to, surgery, radiation therapy, chemotherapy, hormonal therapy, immunotherapy, or molecularly targeted or biologic therapy. Any combination of these other medical treatment methods with a theranostic agent may be used to effectively treat cancer in a subject.

For example, treatment with a theranostic agent may be combined with chemotherapy with one or more chemotherapeutic agents such as, but not limited to, abitrexate, adriamycin, adrucil, amsacrine, asparaginase, anthracyclines, azacitidine, azathioprine, bicnu, blenoxane, busulfan, bleomycin, camptosar, camptothecins, carboplatin, carmustine, cerubidine, chlorambucil, cisplatin, cladribine, cosmegen, cytarabine, cytosar, cyclophosphamide, cytoxan, dactinomycin, docetaxel, doxorubicin, daunorubicin, ellence, elspar, epirubicin, etoposide, fludarabine, fluorouracil, fludara, gemcitabine, gemzar, hycamtin, hydroxyurea, hydrea, idamycin, idarubicin, ifosfamide, ifex, irinotecan, lanvis, leukeran, leustatin, matulane, mechlorethamine, mercaptopurine, methotrexate, mitomycin, mitoxantrone, mithramycin, mutamycin, myleran, mylosar, navelbine, nipent, novantrone, oncovin, oxaliplatin, paclitaxel, paraplatin, pentostatin, platinol, plicamycin, procarbazine, purinethol, ralitrexed, taxotere, taxol, teniposide, thioguanine, tomudex, topotecan, valrubicin, velban, vepesid, vinblastine, vindesine, vincristine, vinorelbine, VP-16, and vumon.

In another example, treatment with a theranostic agent may be combined with targeted therapy with one or more small molecule inhibitors or monoclonal antibodies such as, but not limited to, tyrosine-kinase inhibitors, such as Imatinib mesylate (Gleevec, also known as STI-571), Gefitinib (Iressa, also known as ZD1839), Erlotinib (marketed as Tarceva), Sorafenib (Nexavar), Sunitinib (Sutent), Dasatinib (Sprycel), Lapatinib (Tykerb), Nilotinib (Tasigna), and Bortezomib (Velcade); Janus kinase inhibitors, such as tofacitinib; ALK inhibitors, such as crizotinib; BcI-2 inhibitors, such as obatoclax and gossypol; PARP inhibitors, such as Iniparib and Olaparib; PI3K inhibitors, such as perifosine; VEGF receptor 2 inhibitors, such as Apatinib; AN-152 (AEZS-108) doxorubicin linked to [D-Lys(6)]-LHRH; Braf inhibitors, such as vemurafenib, dabrafenib, and LGX818; MEK inhibitors, such as trametinib; CDK inhibitors, such as PD-0332991 and LEE011; Hsp90 inhibitors, such as salinomycin; small molecule drug conjugates, such as Vintafolide; serine/threonine kinase inhibitors, such as Temsirolimus (Torisel), Everolimus (Afinitor), Vemurafenib (Zelboraf), Trametinib (Mekinist), and Dabrafenib (Tafinlar); and monoclonal antibodies, such as Rituximab (marketed as MabThera or Rituxan), Trastuzumab (Herceptin), Alemtuzumab, Cetuximab (marketed as Erbitux), Panitumumab, Bevacizumab (marketed as Avastin), and Ipilimumab (Yervoy).

In a further example, treatment with a theranostic agent may be combined with immunotherapy, including, but not limited to, using any of the following: a cancer vaccine (e.g., E75 HER2-derived peptide vaccine, nelipepimut-S(NeuVax), Sipuleucel-T), antibody therapy (e.g., Trastuzumab, Ado-trastuzumab emtansine, Alemtuzumab, Ipilimumab, Ofatumumab, Nivolumab, Pembrolizumab, or Rituximab), cytokine therapy (e.g., interferons, including type I (IFNα and IFNβ), type II (IFNγ) and type III (IFNλ) and interleukins, including interleukin-2 (IL-2)), adjuvant immunochemotherapy (e.g., polysaccharide-K), adoptive T-cell therapy, and immune checkpoint blockade therapy.

In a further example, treatment with a theranostic agent may be combined with radiation therapy with a radioisotope, including, but not limited to, iodine-131, strontium-89, samarium-153, and radium-223. In addition, radiation therapy may be combined with administration of a radiosensitizing drug such as, but not limited to, Cisplatin, Nimorazole, and Cetuximab.

### Kits

Kits are also provided for carrying out the methods described herein. In some embodiments, the kit comprises a theranostic agent, described herein, that selectively localizes to metastatic cancerous cells. The kit may further include one or more diagnostic cations such as positron-emitting metal radionuclides suitable for PET imaging such as ⁶⁴Cu, ⁶⁸Ga, ⁴⁴Sc, ⁸⁶Y, ⁸⁹Zr, or ⁸²Rb; gamma-emitting metal radionuclides suitable for SPECT imaging such as ⁶⁷Ga, ^{99m}Tc, ¹¹¹In, or ¹⁷⁷Lu; or paramagnetic metal ions suitable for MRI such as a manganese (e.g., Mn²⁺), iron (e.g., Fe³⁺, Fe²⁺) or gadolinium (e.g., Gd³⁺) cations. In some embodiments, the kit comprises a theranostic agent labeled with a positron-emitting radiohalogen such as ¹²⁴I or ¹⁸F suitable for PET imaging or a gamma-emitting radiohalogen such as ¹³¹I, ¹²⁵I, or ¹²³I suitable for SPECT imaging. In some embodiments, the theranostic agent is conjugated with a radionuclide suitable for radiotherapy such as ¹⁷⁷Lu, ⁹⁰Y, ²¹³Bi, ²¹²Pb, ²¹¹At, ²²⁵Ac, ¹⁶⁶Ho, ¹⁸⁹Sr, ¹⁵³Sm, ²²³Ra, ²²⁶Ra, ¹³⁷Cs, ¹⁹⁸Au, ¹⁸²Ta, ¹⁹²Ir, ¹²⁵I, or ¹³¹I.

Compositions can be in liquid form or can be lyophilized. Suitable containers for the compositions include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle). The kit can further comprise a container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery device. The kit may also provide a delivery device pre-filled with the theranostic agent.

In addition to the above components, the subject kits may further include (in certain embodiments) instructions for practicing the subject methods (i.e., instructions for imaging and treating metastatic cancer with a theranostic agent as described herein). These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), DVD, Blu-ray, flash drive, and the like, on which the information has been recorded. Yet another form of these instructions that may be present is a website address which may be used via the internet to access the information at a removed site.

In one embodiment, the kit comprises a theranostic agent comprising a compound comprising a TMTP1 peptide conjugated to Evans blue (tEB) and DOTA (DOTA-tEB-TMTP1) having the chemical formula:

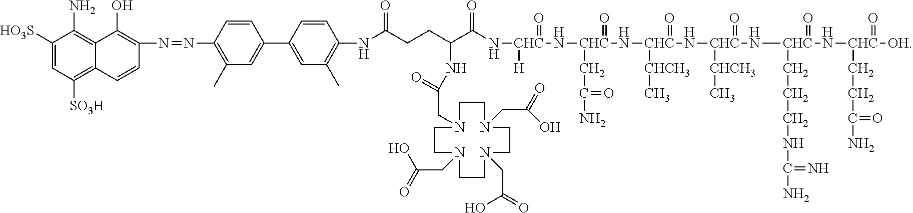

### Utility

The methods described herein are useful for detecting and treating metastatic cancer. In particular, the theranostic agents described herein can be used for imaging and treating a wide variety of cancers, including, but not limited to, breast cancer, ovarian cancer, melanoma, pancreatic cancer, peripheral neuroma, glioblastoma, adrenocortical carcinoma, AIDS-related lymphoma, anal cancer, bladder cancer, meningioma, glioma, astrocytoma, cervical cancer, chronic myeloproliferative disorders, colon cancer, endometrial cancer, ependymoma, esophageal cancer, Ewing's sarcoma, extracranial germ cell tumors, extrahepatic bile duct cancer, gallbladder cancer, gastric cancer, gastrointestinal carcinoid tumors, gestational trophoblastic tumors, hairy cell leukemia, Hodgkin lymphoma, non-Hodgkin lymphoma, hypopharyngeal cancer, islet cell carcinoma, Kaposi sarcoma, laryngeal cancer, leukemia, lip cancer, oral cavity cancer, liver cancer, male breast cancer, malignant mesothelioma, medulloblastoma, Merkel cell carcinoma, metastatic squamous neck cell carcinoma, multiple myeloma and other plasma cell neoplasms, mycosis fungoides and the Sezary syndrome, myelodysplastic syndromes, nasopharyngeal cancer, neuroblastoma, non-small cell lung cancer, small cell lung cancer, head and neck cancer, skin cancer, oropharyngeal cancer, bone cancers, including osteosarcoma and malignant fibrous histiocytoma of bone, paranasal sinus cancer, parathyroid cancer, penile cancer, pheochromocytoma, pituitary tumors, prostate cancer, rectal cancer, renal cell cancer, retinoblastoma, rhabdomyosarcoma, salivary gland cancer, small intestine cancer, soft tissue sarcoma, supratentorial primitive neuroectodermal tumors, pineoblastoma, testicular cancer, thymoma, thymic carcinoma, thyroid cancer, transitional cell cancer of the renal pelvis and ureter, urethral cancer, uterine sarcoma, vaginal cancer, vulvar cancer, and Wilm's tumor and other childhood kidney tumors.

It will be apparent to one of ordinary skill in the art that various changes and modifications can be made without departing from the spirit or scope of the invention.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

### Synthesis of a Long-Circulating Theranostics Agent for Imaging and Treating Metastatic Tumors

### INTRODUCTION

Due to the favorable pharmacokinetics and specific tumor targeting characteristics, peptides are valuable biological tools for tumor imaging and diagnosis, and a number of peptides are currently under investigation. A pentapeptide (NVVRQ) was identified through FliTrx bacterial peptide display system by Ma et al (PLOS One (2012) 7(9): e42685), referred to as TMTP1. TMTP1 displays high affinity for a series of highly metastatic tumor cells. PET is advantageous among nuclear medicine imaging modalities which have been widely used in the diagnosis of cancers. In our previous study, we synthesized an ¹⁸F labeled probe [18F]AIF-NOTA-G-TMTP1, which can specifically target highly metastatic hepatocellular carcinoma, and is suitable for noninvasive visualization of metastatic hepatocellular carcinoma.

### Results

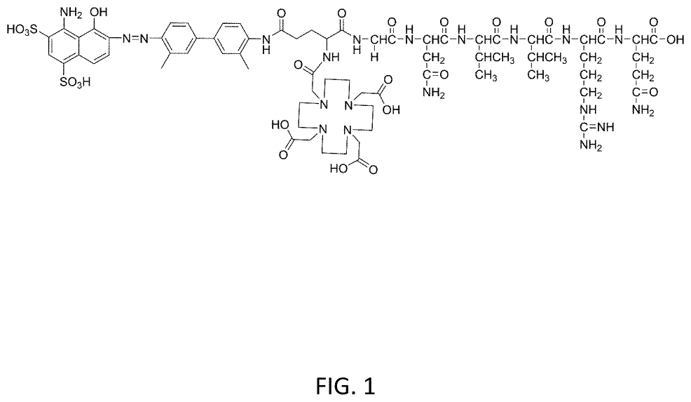

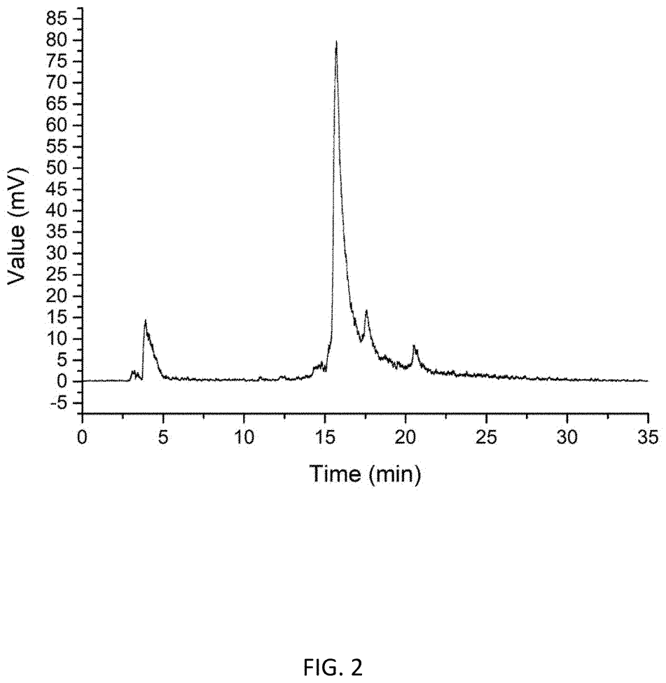

We conjugated TMTP1 with tEB (Evans blue) to synthesize DOTA-tEBTMTP1. The structure of DOTA-EB-TMTP1 is shown in FIG. 1. DOTA-tEB-TMTP1 can be labeled with metal nuclides such as ⁶⁸Ga, ⁶⁴Cu, and ¹⁷⁷Lu. We labeled DOTA-tEB-TMTP1 with ⁶⁴Cu and performed in vitro and in vivo studies. The chemical purity of [⁶⁴Cu]DOTA-EB-TMTP1 was >95% based on analytical HPLC (FIG. 2).

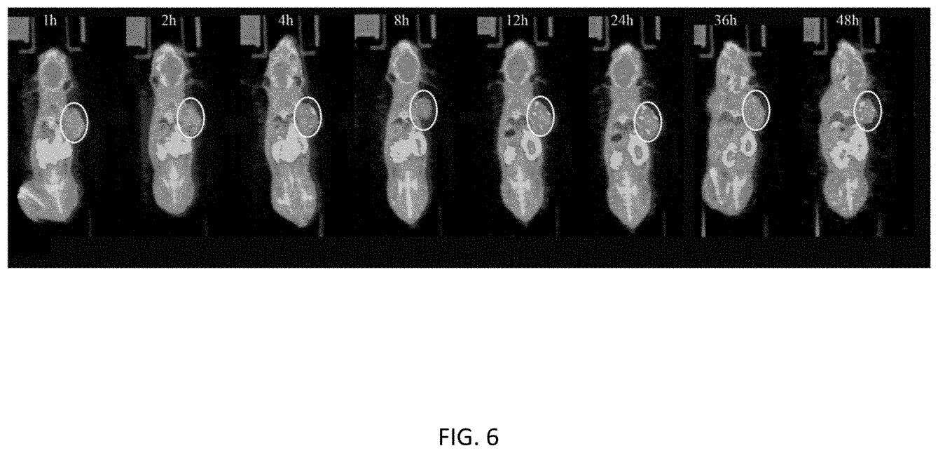

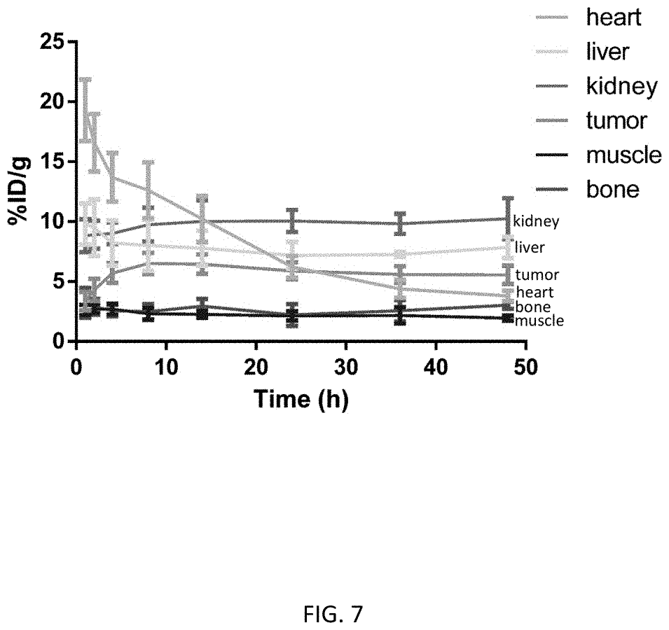

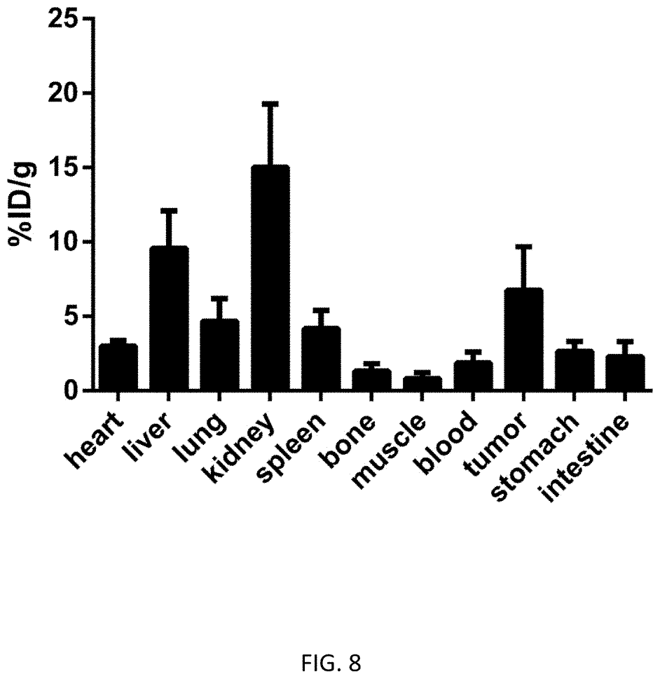

FIG. 6 shows representative whole-body coronal microPET/CT images of 143B tumor bearing nude mice model, which were acquired at 1, 2, 4, 8, 12, 36 and 48 hours after intravenous (i.v.) injection of 3.7-7.4 MBq (100-200 μCO of [⁶⁴Cu]DOTA-EB-TMTP1. The results showed that ⁶⁴Cu-DOTA-tEB-TMTP1 has favorable pharmacokinetic properties. It has a long circulation time in the body and high tumor uptake. Tumor uptake was measured as the percentage of the injected dose per gram of tumor tissue (% ID/g). The PET imaging results showed that the tumor uptake of [⁶⁴Cu]DOTA-EB-TMTP1 increased with time and reached a plateau of 6.5%±0.8% ID/g at 8 hours after injection (FIG. 7). The biodistribution of [⁶⁴Cu]DOTA-EB-TMTP1 showed that the tumor uptake of the probe was still 6.7%±2.9% ID/g at 48 hours after tail vein injection (FIG. 8).

What is claimed is:

1. A theranostic agent comprising a TMTP1 peptide conjugated to a chelating agent and an albumin binding moiety.

2. The theranostic agent of claim 1, wherein the albumin binding moiety.is a truncated Evans blue (tEB) dye, wherein a 1-amino-naphthol-2,4-disulfonic acid moiety of Evans blue is replaced with the chelating agent.

3. The theranostic agent of claim 1, wherein the chelating agent is DOTA (1,4,7,10-tetraazacyclododecane-1,4,7,10-tetraacetic acid).

4. The theranostic agent of claim 3, wherein the theranostic agent comprises a compound having the chemical formula:

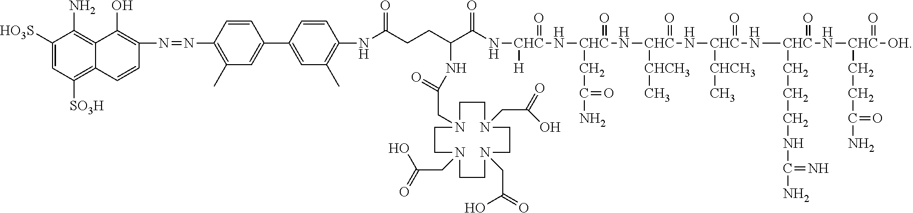

5. The theranostic agent of claim 1, further comprising a detectable label.

6. The theranostic agent of claim 5, wherein the detectable label is a diagnostic metal ion that is chelated by the chelating agent.

7. The theranostic agent of claim 6, wherein the diagnostic metal ion is a metal radionuclide detectable by positron emission tomography (PET) or single photon emission computed tomography (SPECT).

8. The theranostic agent of claim 7, wherein the metal radionuclide is selected from the group consisting of ⁶⁴Cu, ⁶⁷Cu, ⁸⁹Zr, ⁸⁶Y, ⁹⁰Y, ¹¹¹In, ¹⁷⁷Lu, ²⁰¹Tl, ^{99m}Tc, ⁶⁷Ga, ⁶⁸Ga, and ⁸²Rb.

9. The theranostic agent of claim 6, wherein the diagnostic metal ion is a paramagnetic metal ion detectable by magnetic resonance imaging (MRI).

10. The theranostic agent of claim 9, wherein the paramagnetic metal ion is selected from the group consisting of Mn²⁺, Fe³⁺, Fe²⁺, and Gd³⁺.

11. The theranostic agent of claim 1, wherein the theranostic agent is labeled with a radiohalogen.

12. The theranostic agent of claim 11, wherein the radiohalogen is ¹⁸F, ¹³¹I, ¹²⁵I, ¹²⁴I, or ¹²³I.

13. The theranostic agent of claim 1, further comprising an anti-cancer therapeutic agent conjugated to the TMTP1 peptide.

14. The theranostic agent of claim 13, wherein the anti-cancer therapeutic agent is selected from the group consisting of a cytotoxic agent, a drug, a toxin, a nuclease, a hormone, an immunomodulator, a pro-apoptotic agent, an anti-angiogenic agent, a boron compound, a photoactive agent, and a radioisotope.

15. The theranostic agent of claim 14, wherein the radioisotope is ¹⁷⁷Lu, ⁹⁰Y, ²¹³Bi, ²¹²Pb, ²¹¹At, ²²⁵Ac, ¹⁶⁶Ho, ¹⁸⁹Sr, ¹⁵³Sm, ²²³Ra, ²²⁶Ra, ¹³⁷Cs, ¹⁹⁸Au, ¹⁸²Ta, ¹⁹²Ir, ¹²⁵I, or ¹³¹I.

16. A composition comprising the theranostic agent of claim 1 and a pharmaceutically acceptable excipient.

17. The composition of claim 16, further comprising an anti-cancer therapeutic agent.

18. The composition of claim 17, wherein the anti-cancer therapeutic agent is selected from the group consisting of a chemotherapeutic agent, an immunotherapeutic agent, a biologic therapeutic agent, a pro-apoptotic agent, an angiogenesis inhibitor, a photoactive agent, a radiosensitizing agent, and a radioisotope.

19. A kit comprising the composition of claim 16 and instructions for using the kit for detecting and treating metastatic cancer.

20. A method of imaging cancerous tissue of a patient suspected of having metastatic cancer, the method comprising:
a) contacting tissue of the patient suspected of being cancerous with a detectably effective amount of the composition of claim 16 under conditions wherein metastatic cancerous cells, if present, in the tissue uptake the theranostic agent; and
b) imaging the tissue of the patient, wherein detection of increased uptake of the theranostic agent into the tissue of the patient compared to a control indicates that the patient has metastatic cancer.

21. The method of claim 20, wherein the tissue is contacted with the theranostic agent in vivo or in vitro.

22. The method of claim 20, wherein said imaging is performed using positron emission tomography (PET), single photon emission computed tomography (SPECT), magnetic resonance imaging (MRI), positron emission tomography-computed tomography (PET-CT), positron emission tomography-magnetic resonance imaging (PET-MRI), or single photon emission computed tomography-magnetic resonance imaging (SPECT-MRI).

23. A method of monitoring progression of metastatic cancer in a patient, the method comprising: imaging tissue of the patient according to the method of claim 20, wherein a first image is obtained at a first time point and a second image is obtained later at a second time point, wherein detection of increased uptake of the theranostic agent into the tissue of the patient at the second time point compared to the first time point indicates that the patient is worsening, and detection of decreased uptake of the theranostic agent into the tissue of the patient at the second time point compared to the first time point indicates that the patient is improving.

24. The method of claim 23, wherein increased uptake of the theranostic agent into the tissue of the patient is associated with growth of a tumor or presence of more tumors or metastatic cancerous cells at the second time point.

25. A method for evaluating the effect of an agent for treating cancer in a patient, the method comprising: imaging tissue of the patient according to the method of claim 20 before and after the patient is treated with said agent, wherein detection of increased uptake of the theranostic agent into the tissue of the patient after the patient is treated with said agent compared to before the patient is treated with said agent indicates that the patient is worsening, and decreased uptake of the theranostic agent into the tissue of the patient after the subject is treated with said agent compared to before the patient is treated with said agent indicates that the patient is improving.

26. A method for treating cancer comprising administering to a subject in need thereof a therapeutically effective amount of the composition of claim 16.

27. The method of claim 26, further comprising monitoring uptake of the theranostic agent into metastatic cancerous cells of the subject by detecting the theranostic agent.

28. The method of claim 27, further comprising recording one or more images of metastatic cancerous cells that uptake the theranostic agent in the subject.

29. The method of claim 26, further comprising monitoring anti-tumor activity of the theranostic agent by recording one or more images of the metastatic cancerous cells that uptake the theranostic agent in the subject.

30. The method of claim 28, where the one or more images are obtained using positron emission tomography (PET), single photon emission computed tomography (SPECT), magnetic resonance imaging (MRI), positron emission tomography-computed tomography (PET-CT), positron emission tomography-magnetic resonance imaging (PET-MRI), or single photon emission computed tomography-magnetic resonance imaging (SPECT-MRI).

31. The method of claim 26, further comprising administering an anti-cancer therapeutic agent.

32. The method of claim 31, wherein the anti-cancer therapeutic agent is selected from the group consisting of a chemotherapeutic agent, an immunotherapeutic agent, a biologic therapeutic agent, a pro-apoptotic agent, an angiogenesis inhibitor, a photoactive agent, a radiosensitizing agent, and a radioisotope.
